(19) United States
(12) Patent Application Publication
(10) Pub. No.: US 2022/0130489 A1
(43) Pub. Date: Apr. 28, 2022

(54) SYSTEM AND METHOD FOR PROVIDING NEOANTIGEN IMMUNOTHERAPY INFORMATION BY USING ARTIFICIAL-INTELLIGENCE-MODEL-BASED MOLECULAR DYNAMICS BIG DATA

(71) Applicant: SYNTEKABIO,INC., Daejeon (KR)

(72) Inventors: Jongsun JUNG, Daejeon (KR); Jonghui HONG, Seoul (KR)

(73) Assignee: SYNTEKABIO,INC., Daejeon (KR)

(21) Appl. No.: 17/438,822

(22) PCT Filed: Mar. 12, 2020
(86) PCT No.: PCT/KR2020/003464

§ 371 Date: Sep. 13, 2021

(30) Foreign Application Priority Data
Mar. 12, 2019 (KR) 10-2019-0028278
Apr. 5, 2019 (KR) 10-2019-0040367
Mar. 12, 2020 (KR) 10-2020-0030597

(51) Int. Cl. G16B 20/20; G01N 33/68; G16B 5/00; G16B 40/00

(52) U.S. Cl. CPC G16B 20/20; G16B 40/00; G16B 5/00; G01N 33/68

(57) ABSTRACT

Disclosed are a system and a method of predicting neoantigens and immune response induction. The system and the method may verify induction of immunity against neoantigens having high binding affinity by identifying neoantigen candidates through genomic mutations and then predicting the binding affinities of the neoantigen candidates for MHC through molecular dynamics. The method provides neoantigen immunotherapy information for identifying a neoantigen using artificial intelligence (AI)-based molecular dynamics big data, and includes steps of: (A) identifying neoantigen candidates through a genomic mutation; (B) filtering the specificities of the neoantigen candidates for tissue and disease; (C) predicting the in silico binding of the neoantigens to MHC; and (D) calculating and ranking TCR activity.

### CROSS-REFERENCE TO PRIOR APPLICATIONS

This application is a National Stage Patent Application of PCT International Patent Application No. PCT/KR2020/003464 (filed on Mar. 12, 2020) under 35 U.S.C. § 371, which claims priority to Korean Patent Application Nos. 10-2019-0028278 (filed on Mar. 12, 2019), 10-2019-0040367 (filed on Apr. 5, 2019), and 10-2020-0030597 (filed on Mar. 12, 2020), which are all hereby incorporated by reference in their entirety.

### BACKGROUND

The present disclosure relate to an immunotherapy system and method of identifying neoantigens using AI-based molecular dynamics big data, and more particularly, to a system and a method of predicting neoantigens and immune response induction based on molecular dynamics, the system and the method making it possible to verify induction of immunity against neoantigens having high binding affinity by identifying neoantigen candidates through genomic mutations and then predicting the binding affinities of the neoantigen candidates for MHC.

Cancer is known to show mutations in hundreds of genes during the development and proliferation thereof. Major oncogenes have mutation sites at more than 10 positions, and these mutations vary in frequency and type of variant sequence depending on the carcinoma and patient. These mutations lead to specific amino acid sequence changes through transcripts (RNA transcripts), and eventually produce peptides (neoantigens). Thus, all types of cancer cells express neoantigens in the form of cancer cell-specific peptides, and when these peptides (neoantigens) bind to MHC I on the cancer cell surface, T-cell receptor (TCR) selectively recognizes these peptides and induces anticancer immunity.

In particular, it is known that cancer cell-specific peptides (neoantigens) have high specificity for cancer cells, unlike tumor-associated antigens such as overexpressed antigens or cancer/testis antigens, but do not cause problems such as immune tolerance or autoimmunity. Thus, these are used as a main target for T cell-based cancer immunotherapy. Meanwhile, more than 130 therapeutic agents based on cancer cell-specific neoantigens have been developed as cell therapy products or peptide-based cancer vaccines, and the anticancer effects thereof have been gradually demonstrated through clinical trials for various carcinomas in cancer patients. Immunomodulatory cell therapy products are divided, according to the immune cells used and the characteristics of genes introduced into cells in the production process, into dendritic cells, lymphokine activated killer (LAK) cells, T cell-based immunomodulatory therapy products (tumor-infiltrating T lymphocytes (TILs)), T cell receptor-modified T (TCR-T) cells, and chimeric antigen receptor-modified T (CAR-T) cells. T cells have the advantage of selectively recognizing only tumor cells, whereas TCR-T and TILs have the advantage of being able to target antigens inside tumor cells as well as the tumor surface. Thus, studies on immunomodulatory cell therapy based on neoantigens are expected to be more active. Peptide-based cancer vaccines are classified, based on the frequency of mutation, into two types: shared neoantigens (for off-the-shelf treatment) for hot spot mutations of major oncogenes; and private neoantigens (for personalized treatment) that appear only in specific patients. These vaccines are injected into patients in the form of a pool set of about 10 neoantigens to enhance anticancer efficacy.

Thus, when patient-specific neoantigens containing specific mutations are identified, they are applicable to patient-specific immunotherapy regardless of the type of cancer.

Recent advances in NGS technology made it possible to identify mutations in individual human genome sequences within a short time based on information about genomic mutations present in tumor exomes and tumor transcriptomes that appear in cancer patient biopsies. As a result, the opportunities for identifying neoantigens have increased dramatically, and bioinformatic neoantigen prediction technologies such as TSNAD, pVAC-Seq, and INTERGRATE-neo, which are based on such genomic information, have been developed.

Nevertheless, the development of anticancer immune vaccines based on neontigens still has problems of high cost and relatively low efficiency. Major histocompatibility complex (MHC) proteins to which neoantigens on the cancer cell surface bind are broadly divided into MHC I and MHC II, which are subdivided into HLA-A, HLA-B, HLA-C, HLA-DR, HLA-DP and HLA-DQ. The total number of alleles for each of these proteins has been found to be ten thousand or more, and the type and number of immunotypes expressed in each individual are very diverse. In addition, only a very small number of mutations in expressed mutant proteins may be recognized as antigens by T cells. Thus, efficient identification of neoantigens showing immunogenicity in each patient can be considered a key factor in the success or failure of developing anticancer immune vaccines. To this end, it is very important to develop technology of increasing the predictive ability of identifying neoantigens.

The present disclosure proposes a molecular dynamics-based system (NeoScan) and method for predicting neoantigens and induction of immune response, which can efficiently verify induction of immunity against neoantigens having high binding affinity by predicting the binding affinities of various neoantigen candidates for MHC through AI-based molecular dynamics big data analysis on the basis of the three-dimensional structures of immono-subtype proteins.

### SUMMARY

The present disclosure is intended to construct a patient-specific neoantigen prediction platform using a tumor-specific cumulative mutation prediction technology developed by prior research, and to treat a disease by an immunotherapy method using the same.

Moreover, the present disclosure is intended to commercialize a platform that predicts a number of tumor-specific neoantigens based on a patient-specific genome-transcript-protein or the like.

Furthermore, the present disclosure is intended to realize medical industrialization through a cancer patient-specific neoantigen prediction platform using AI deep learning-combined precision medical technology based on big data.

In addition, the present disclosure is intended to use neoantigens, predicted based on NEOscan, for immunity induction verification tests, immunotherapy products, and cell therapy products based on T cell receptor-modified T (TCR-T) cells, chimeric antigen receptor-modified T (CAR-T) cells and tumor-infiltrating T lymphocytes (TIL).

According to the features of the present disclosure for achieving the above-described objects, the present disclosure provides a neoantigen immunotherapy system and method, which identify a highly immunogenic neoantigen using NEOscan technology, which is a system of predicting neoantigens using an artificial intelligence (AI) model-based molecular dynamics big data to identify patient cancer cell-specific neoantigens, based on information about a genomic mutation present in tumor exomes and tumor transcriptomes that appear in cancer patient biopsies, and use the identified neoantigens for immunotherapy of major human diseases including cancer.

Here, the genomic mutation present in the tumor exomes and transcriptomes may be any one of neo mutations, exposed features or mal-functions, and verification of exome and transcriptome expression may be performed by determining over-expression and differential expression in the transcriptome.

Moreover, determination of an immunotype to which a neoantigen identified by NEOscan binds may be performed by determining that the immunotype of the cancer patient is any one of HLA-A, HLA-B, HLA-C, HLA-DR, HLA-DP or HLA-DQ.

Furthermore, the present disclosure may further include predicting the binding affinities of neoantigens for MHC; and the binding affinities of neoantigens for MHC may be determined by generating binding models for different types of antigens and calculating the energy difference and RMSD difference therebetween.

In addition, the present disclosure may further include predicting immune response induction; and the predicting of the immune response induction may be performed by determining whether the amino acid types at specific positions (p1 to p9) of the antigen are expressed.

In addition, the present disclosure further includes inducing immunity against a neoantigen for which immune response induction has been predicted; and the inducing of the immunity may be performed by any one of VLP, adjuvant, modification, stimulation or inhibition.

In addition, according to the present disclosure, the neoantigen against which the induction of immunity has been confirmed may be applied to vaccines and therapeutic drugs for treatment of all types of cancers and other diseases resulting from human genomic mutations.

### BRIEF DESCRIPTION OF THE DRAWINGS

FIG. 1 is a flow diagram showing a neoantigen-specific treatment process according to the present disclosure.

FIG. 2 is a conceptual view showing selecting major clone genes from cancer cells according to the present disclosure.

FIG. 3 is a conceptual view showing the functional relationship between mesenchymal stroma cells (MSC) and cancer cell proliferation according to the present disclosure.

FIG. 4 is a conceptual view showing a structure for calculating six HLA genotypes based on genomic NGS according to the present disclosure.

FIG. 5 illustrates a heat-map of disease-associated tissues according to the present disclosure.

FIG. 6 is a conceptual view showing a process of calculating in silico binding affinity (IBA) based on dynamics simulation according to the present disclosure.

FIG. 7 illustrates a portion of a dynamics simulation process in the in silico binding affinity (IBA) calculation process according to the present disclosure.

FIG. 8 illustrates a portion of the results of in silico binding affinity (IBA) calculation according to the present disclosure.

FIG. 9 illustrates an example of a peptide phi-psi angle-based Ramachandran plot for in silico binding affinity (IBA) according to the present disclosure.

FIG. 10 shows the correlation between phi-psi angle and structure rmsds according to the present disclosure.

FIG. 11 shows the correlation between selected features and structure rmsds according to the present disclosure.

FIG. 12 is a conceptual view showing a feature-based AI model structure generated from MHC-peptide complexes according to the present disclosure.

FIG. 13 shows the results of AI deep learning between selected features and structure rmsds according to the present disclosure.

FIG. 14 shows an example of ranking TCR activity according to the present disclosure.

FIG. 15 is a table showing the results of verifying the in silico binding affinities (IBA) of neoantigens, predicted according to the present disclosure, for HLA-A*2402 by a testing institution (PROIMMUNE).

FIG. 16 is a table showing the results of verifying the in silico binding affinities (IBA) of neoantigens, predicted according to the present disclosure, for HLA-A*0201 by a testing institution (PROIMMUNE).

FIG. 17 is a table showing the results of verifying the in silico binding affinities (IBA) of neoantigens, predicted according to the present disclosure, for HLA-A*11:01 by a testing institution (PROIMMUNE).

### DETAILED DESCRIPTION

A preferred embodiment of the present disclosure is directed to a method and a system of providing neoantigen immunotherapy information for identifying neoantigens using artificial intelligence (AI)-based molecular dynamics big data, the method including steps of: (A) identifying neoantigen candidates through a genomic mutation; (B) filtering the specificities of the neoantigen candidates for tissue and disease; (C) predicting the in silico binding of the neoantigens to MHC; and (D) calculating and ranking TCR activity.

Here, the genomic mutation is a mutation present in tumor exomes and tumor transcriptomes that appear in cancer patient biopsies.

Meanwhile, the mutation present in tumor exomes and tumor transcriptome may be any one of neo mutations, exposed features or mal-functions, and verification of exome and transcriptome expression may be performed by determining over-expression and differential expression in the transcriptomes.

Moreover, determination of an immunotype to which a neoantigen identified by NEOscan bind may be performed by determining that the immunotype of the cancer patient is any one of HLA-A, HLA-B, HLA-C, HLA-DR, HLA-DP or HLA-DQ.

Furthermore, the binding affinities of neoantigens for MHC may be determined by generating binding models for different types of antigens and calculating the energy difference and RMSD difference therebetween.

In addition, the predicting of immune response induction may be performed by determining whether the amino acid types at specific positions (p1 to p9) of the neoantigen are expressed.

In addition, the present disclosure further includes inducing immunity against a neoantigen for which immune response induction has been predicted; and the inducing of the immunity may be performed by any one of VLP, adjuvant, modification, stimulation or inhibition.

In addition, according to the present disclosure, the neoantigen against which the induction of immunity has been confirmed may be applied to vaccines and therapeutic drugs for treatment of all types of cancers and other diseases resulting from human genomic mutation.

Hereinafter, the neoantigen immunotherapy system and method using artificial intelligence (AI)-based molecular dynamics big data according to specific embodiments of the present disclosure will be described with reference to the accompanying drawings.

The effects and features of the present disclosure, and the way of attaining them, will become apparent with reference to the exemplary embodiments described below in detail along with the accompanying drawings. However, the present disclosure is not limited to the exemplary embodiments disclosed below and can be embodied in a variety of different forms; rather, these embodiments are provided so that this disclosure will be thorough and complete, and will fully convey the scope of the present disclosure to those skilled in the art to which the present disclosure pertains. The scope of the present disclosure will be defined only by the appended claims.

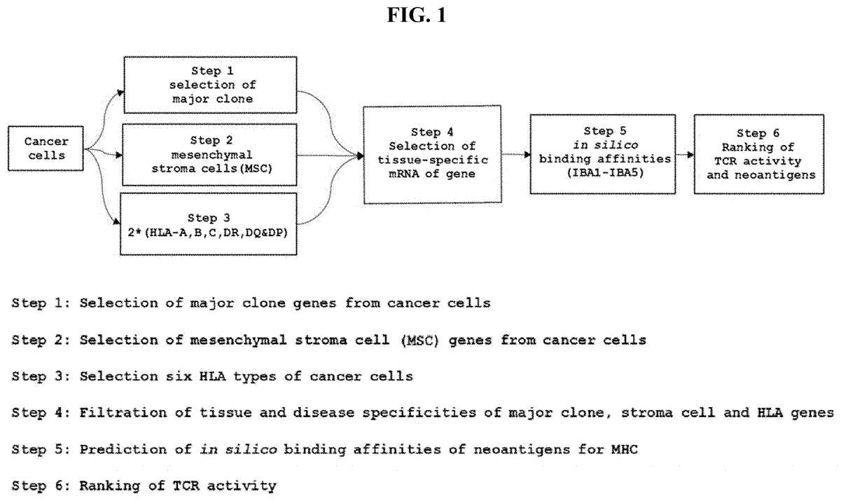

First, FIG. 1 shows a neoantigen-specific treatment method according to the present disclosure. As shown therein, the neoantigen-specific treatment method according to the present disclosure includes steps: (1) selecting major clone genes from cancer cells; (2) selecting mesenchymal stroma cell (MSC) genes from the cancer cells; (3) selecting six HLA types of the cancer cells; (4) filtering the tissue/disease specificities of the major clones, the stroma cells and the HLA genes; (5) predicting the in silico binding affinity of neoantigens to MHC; and (6) ranking TCR activity.

As shown in FIG. 1, in step (1), a major clone having the largest number of cancer cells is selected from cancer cells composed of the major clone and secondary and tertiary subclones, and gene mutations found in the major clone are collected.

In step (2), it is required to collect neoantigens based on somatic mutation of genes expressed in stroma cells, because mesenchymal stroma cells (or stroma) are involved in the proliferation of cancer cells.

In step (3), it is possible to determine the type of the individual by genomic HLA typing, and considering the heterotypes of six relevant HLA genes, it is necessary to predict the type of up to 12 genotypes.

In step (4), whether the major clone genes of cancer, the mesenchymal stroma cell (stroma) somatic genes, and the six major HLA genes are expressed in a specific tissue is confirmed.

In step (5), based on the three-dimensional structures of the somatic mutation-based peptides of the tissue-specific genes generated through step (1) to step (4) and the selected MHC protein, in silico binding affinity (IBA) is calculated,

Finally, in step (6), ranking is determined through calculation of amino acid position-specific TCR activity for final neoantigens selected based on the in silico binding affinity (IBA).

In the present disclosure, 10 or more neoantigens are generated for each patient through the above-described process.

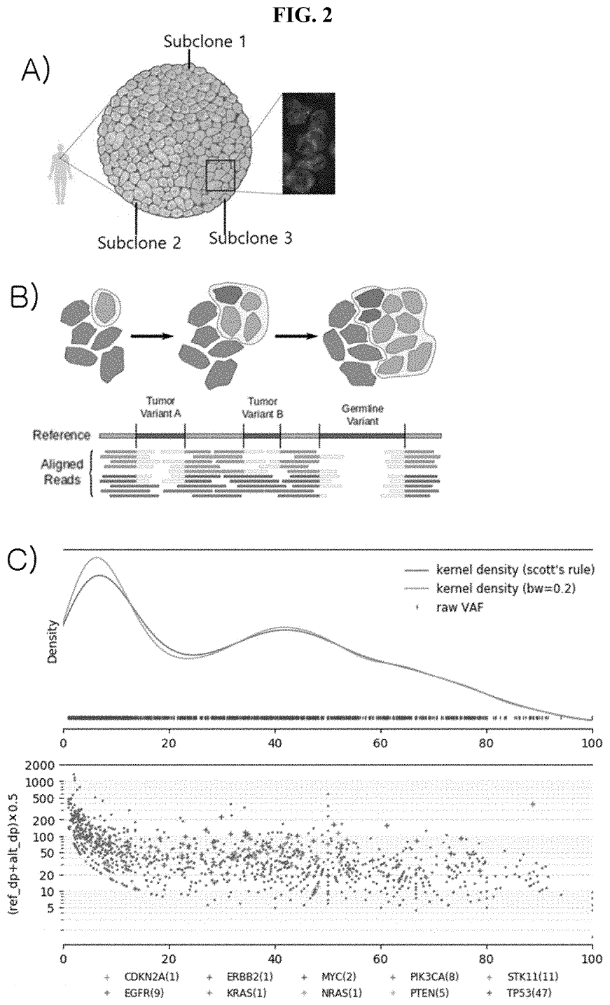

FIG. 2 illustrates a method for selecting major clone genes from cancer cells according to the present disclosure. This selection method was developed in-house by the applicant and is hereinafter referred to as “driver mutation scanning”.

Hereinafter, the driver mutation scanning will be described.

FIG. 2(A) shows an example in which subclone 1, subclone 2 and subclone 3 are included in a tissue having specific human cancer cells. FIG. 2(B) shows a clone by a schema (structure definition) and shows that genomic sequence fragments are aligned to predict a clone. FIG. 2(C) shows a kernel density plot (X-axis: VAF % (variant allele frequency)) which serves as a basis for clonal evolution including “driver markers of EGFR gene” predicted according to the present disclosure. In addition, FIG. 2(C) shows that an EGFR driver marker belongs to the first subclone among four subclones.

In addition, FIG. 2(C) shows a kernel density plot (X-axis: VAF % (variant allele frequency), and Y-axis: value obtained by Ref depth plus Alt depth by 2) which serves as a basis for two big clonal evolutions including the driver markers extracted from samples used in about 150 training courses, and FIG. 2(C) shows known or newly predicted driver markers. In particular, VAF %>5, and known driver and predicted driver mutations and the numbers thereof are marked with the symbol “+” along with the gene names.

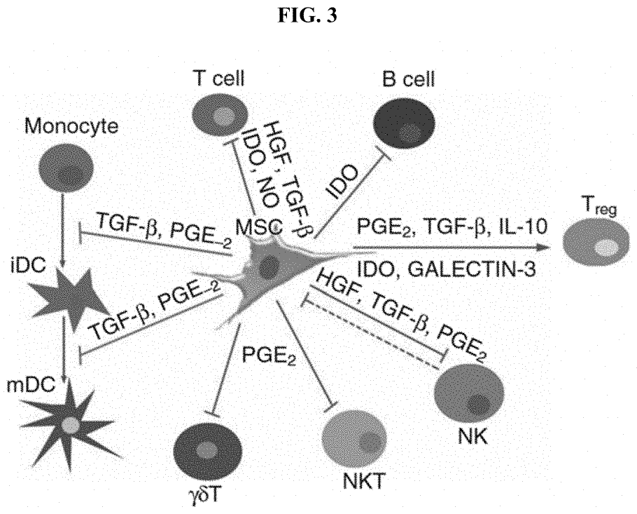

FIG. 3 shows the functional relationship between mesenchymal stroma cell (MSC) and cancer cell proliferation.

As shown in FIG. 3, mesenchymal stroma cells (or stroma) are involved in cancer cell proliferation, and hence it is required to collect neoantigens based on somatic mutation of genes expressed in the stroma cells.

Here, whether stroma cells are present in cancer cells is confirmed by the public tool ESTIMATE method proposed by MD Anderson Hospital of Texas State University and a variety of similar methods.

The ESTIMATE may be applied to evaluate the presence of stroma cells in tumor samples and filtration of immune cells using gene expression data. This method is publicly available through the SourceForge public software repository (https://sourceforge.net/projects/estimateproject/).

When the ESTIMATE is applied to a new microarray or RNA-seq based transcriptome profile as well as a publicly available microarray expression data set, it helps to reveal the role of neocytes in microenvironments and provide new information about the situation in which genomic changes occur.

FIG. 3 shows a schema (structure) for the functional relationship between mesenchymal stroma cells (MSCs) and cancer cell proliferation. The schema shows the effect of mesenchymal stroma cells (MSCs) on immune cells.

MSCs regulate immune response by interaction with a wide range of immune cells, including T cells, B cells, dendritic cells (DCs), regulatory T cells (T), natural killer (NK) cells, NK T cells and γδ T cells.

The inhibitory role of MSCs depends on cell-cell contact and soluble factors released by MSCs.

The abbreviations used in FIG. 3 denote the followings: HGF: hepatocyte growth factor; iDC: immature dendritic cells; IDO: indoleamine 2,3-dioxygenase; IL-10: interleukin-10; mDC: mature dendritic cells; NO: nitric oxide; PGE2: prostaglandin E2; TGF-b: transforming growth factor-b. (Ref: Clinical and Experimental Immunology, 164: 1-8, 2011).

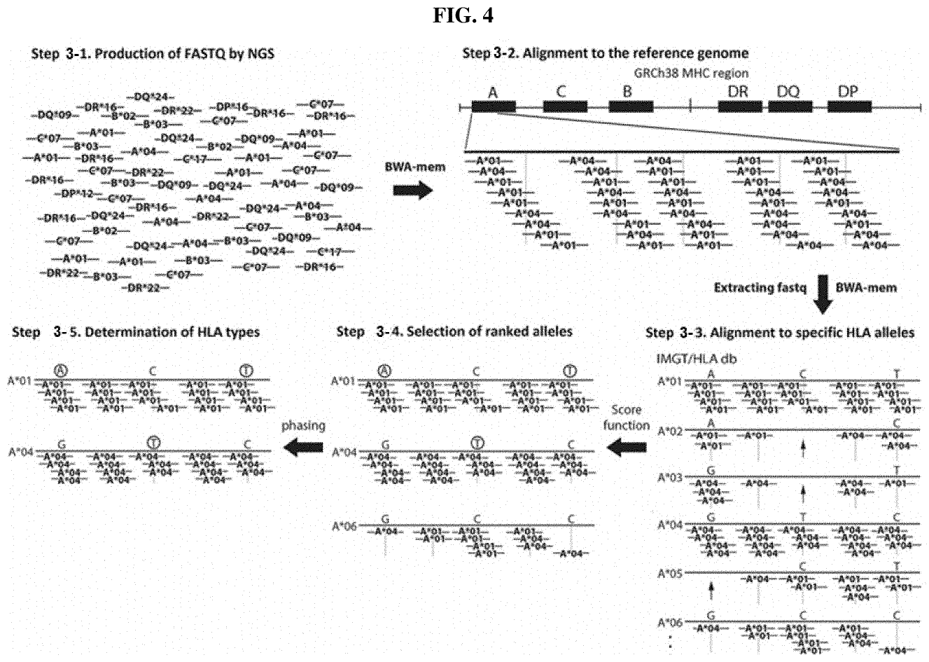

Meanwhile, FIG. 4 shows a schema (structure) for calculating six HLA genotypes based on genomic NGS according to the present disclosure.

As shown in FIG. 4, HLAscan performs alignment of reads to HLA sequences from the International ImMunoGeneTics Project/Human Leukocyte Antigen (IMGT/HLA) database. In addition, the distribution of aligned reads is used to calculate a score function to determine correctly phased alleles by progressively removing false-positive alleles.

Comparative HLA typing tests using public datasets from the 1000 Genomes Project and the International HapMap Project demonstrate that HLAscan can perform HLA typing more accurately than previously reported NGS-based methods such as HLAscaner and PHLAT.

In addition, it is confirmed that the results of HLA-A, -B, and -DRB1 typing, predicted by HLAscan using data generated by NextGen, are identical to those obtained using a Sanger sequencing-based method.

In addition, the present inventors applied HLAscan to a family dataset with various coverage depths generated on the Illumina HiSeq X-TEN platform. As a result, HLAscan identified allele types of HLA-A, -B, -C, -DQB1, and -DRB1 with 100% accuracy for sequences at ≥90× depth, and the overall accuracy was 96.9%.

This method is described in detail in U.S. Pat. No. 10,540,324 B2 owned by the applicant.

Meanwhile, FIG. 4 shows a schema for calculating six HLA genotypes based on genomic NGS. The algorithm of HLAscan is explained schematically in five main steps.

That is, the process of step (3) described above with reference to FIG. 1 is performed by the following detailed process. As shown in FIG. 4, step 3-1 shows collection of read sequences of HLA genes produced from a sample.

Step 3-2 demonstrates alignment of the HLA-A gene read sequence to the human reference genome sequence.

Step 3-3 shows a process in which HLA-A gene read sequences are aligned to specific allele types, and step 3-4 shows a process in which ranked alleles are selected.

In steps 3-3 and 3-4, HLA-A read sequences are aligned to specific allele types. From the candidate alleles, true allele types are determined by applying a score function (step 3-3 and step 3-4).

Step 3-5 shows a process of determining HLA types.

In FIG. 4, the arrows under reference sequences represent positions with sequence variance. Black arrows in alleles A*02, A*03, and A*05 of step 3-3 indicate genetic positions with no sequence reads aligned. In addition, circled bases in step 3-4, A and T in A*01, and T in A*04 represent unique sequences that are not redundant with base sequences in other ranked alleles (Ref.: Ka et al., BMC Bioinformatics (2017), 18:258).

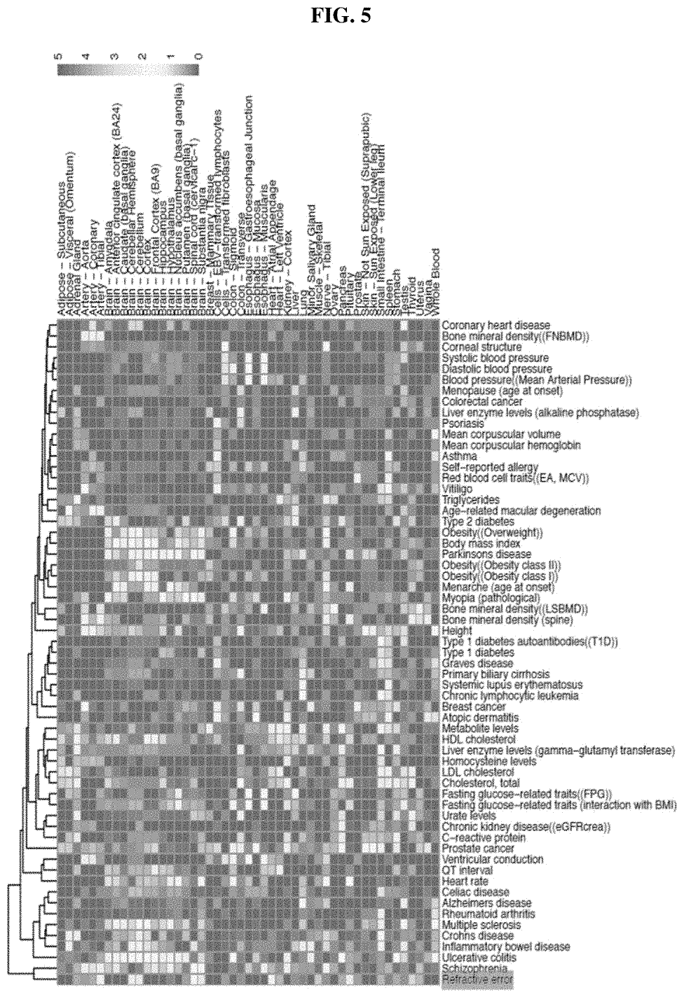

FIG. 5 shows a heat-map of disease-associated tissues according to the present disclosure.

The heat-map of disease-associated tissues shown in FIG. 5 confirms that, in step 3-4 described above, the major clone genes of cancer, mesenchymal stroma cell (stroma) somatic cell genes, and six major HLA genes, are expressed in specific tissues.

To derive such results, the public results in the international consortium paper are applied to determine the tissue/disease specificity of genes. In the corresponding paper, in order to determine the tissue/disease specificity of genes, 8,527 high-quality RNA-seq samples covering 36 human peripheral tissues and 13 brain subregions were collected, and data obtained by calculating tissue-specific gene expression therefrom were published.

The corresponding paper is “A systematic survey of human tissue-specific gene expression and splicing reveals new opportunities for therapeutic target identification and evaluation,” BioRxiv, 2018 (https://doi.org/10.1101/311563).

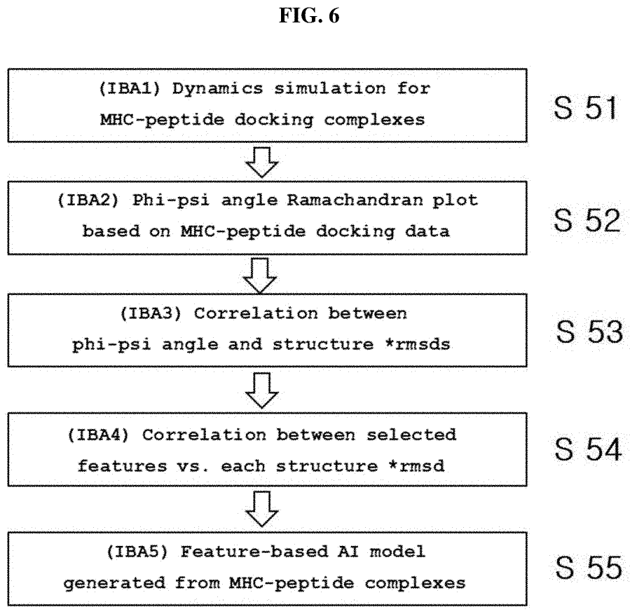

FIG. 6 shows a process of calculating in silico binding affinity (IBA) based on dynamics simulation according to the present disclosure.

That is, in step (5) described above, in silico binding affinity (IBA) is calculated through three-dimensional structure-based docking of MHC protein to somatic mutation-based peptides of tissue-specific genes generated through steps (1) to (4).

Specifically, as shown in FIG. 6, dynamics simulation for MHC-peptide docking complexes is performed (S51), and a phi-psi angle Ramachandran plot is generated based on the MHC-peptide docking data (S52).

Then, the correlation between phi-psi angle and structure rmsds is calculated (S53). Next, the correlation between the selected feature and each structure rmsd is calculated (S54), and binding affinity is finally determined through an AI model generated based on the features generated from the MHC-peptide complexes (S55).

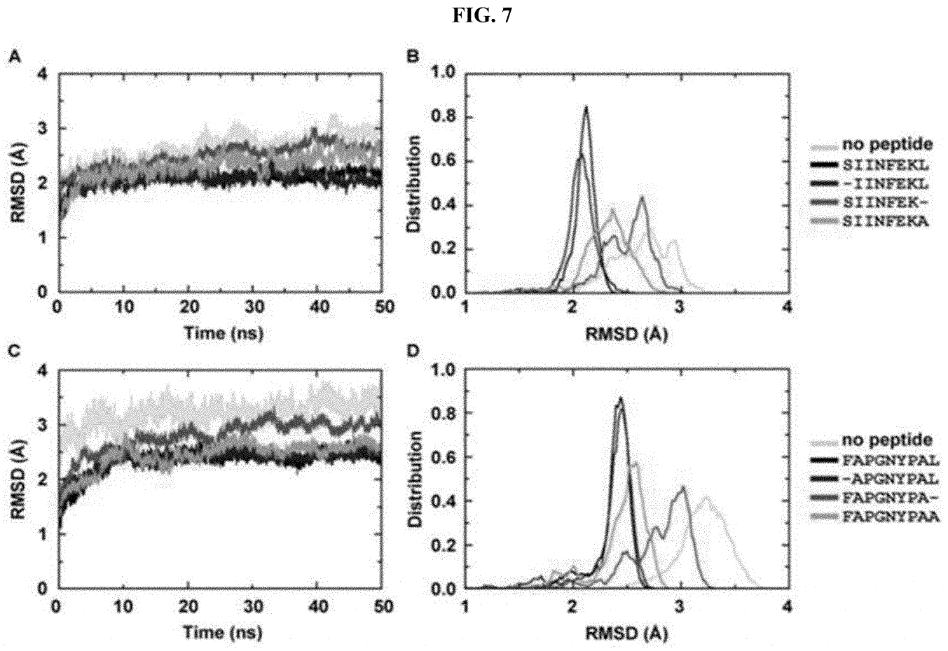

Meanwhile, FIG. 7 shows a portion of the dynamics simulation process of the in silico binding affinity (IBA) calculation process according to the present disclosure.

The in silico binding affinity (IBA) shown in FIG. 7 is calculated according to the following equation:

IBA=log(pred_mutant_ic50)/log(pred_wild_type_ic50).

Here, IBA>1 means binding, and the ratio of mutant to wildtype and examples of simulation of p1-deletion and p9-deletion models for various comparisons are shown.

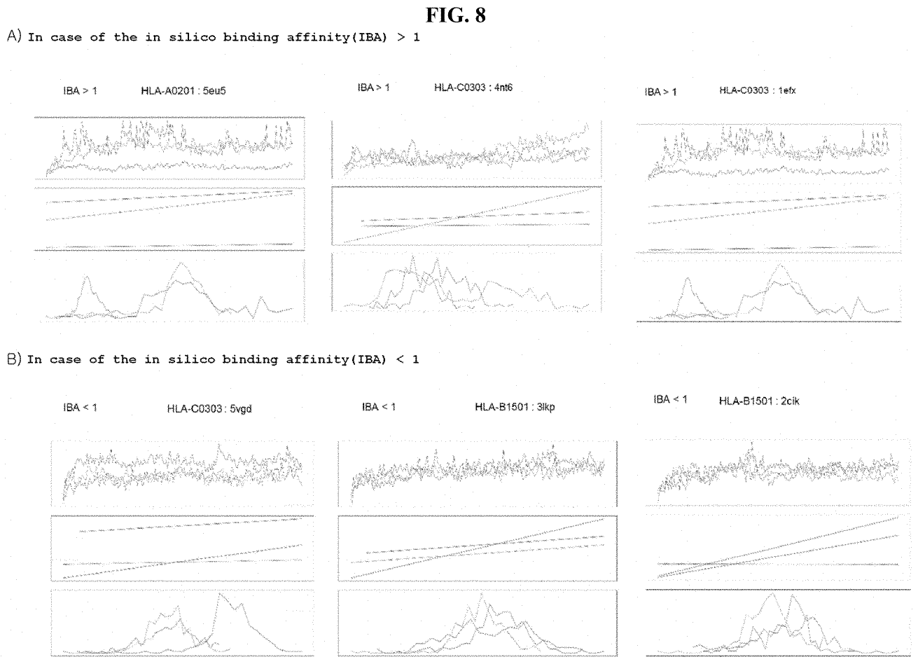

FIG. 8 shows the results of calculating in silico binding affinity (IBA) according to the present disclosure.

Specifically, FIG. 8(A) shows the results obtained when in silico binding affinity (IBA) is greater than 1, and shows result values for HLA-A0201 (5eu5), HLA-00303 (4nt6) and HLA-C0303 (lefx). FIG. 8(B) shows the results obtained when in silico binding affinity (IBA) is smaller than 1, and shows result values for HLA-00303 (5vgd), HLA-B1501 (31kp) and HLA-B1501 (2cik).

That is, FIG. 8 shows a practical example of step (51), which is dynamics simulation for in silico binding affinity (IBA). Here, the IBA ratio is calculated according to the following equation:

IBA ratio=log(pred_mutant_ic50)/log(pred_wild_type_ic50).

Here, IBA>1 means binding, and the score according to the magnitude of the ratio is applied differentially.

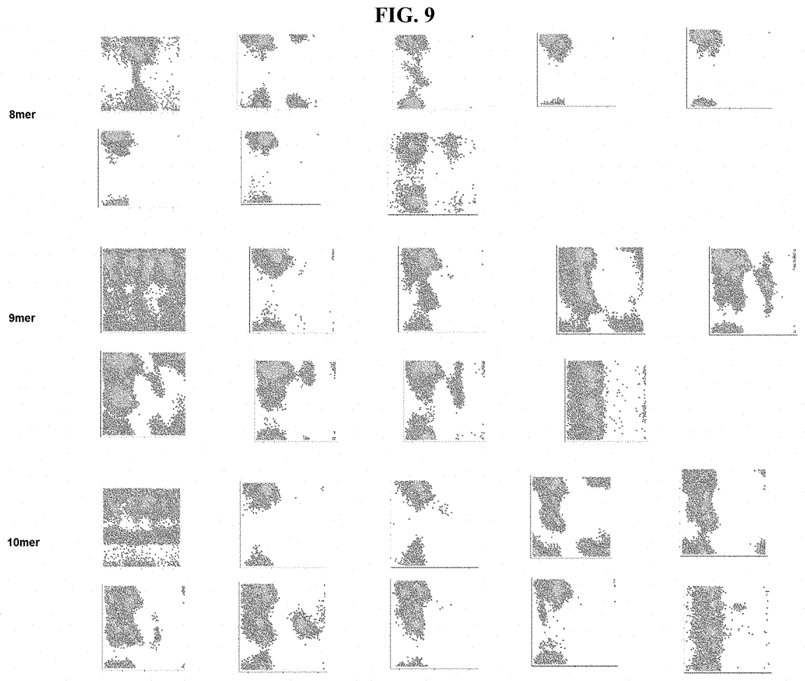

Meanwhile, FIG. 9 shows an example of a peptide phi-psi angle-based plot for in silicon binding affinity (IBA) according to the present disclosure.

As shown in FIG. 9, to show step (52) which is the second step of IBA calculation, an angle-based Ramachandran plot at each peptide amino acid position is shown for 1,000 moving snapshots of each of peptides 8-mer, 9-mer and 10-mer. Here, the x-axis is phi, the y-axis is psi.

In FIG. 9, internal different color dots among dots show the angle of the docking occurrence (*rmsd <1) structure. Here, *rmsd means the root mean square deviation of the coordinates between the correct answer structure and the docking structure.

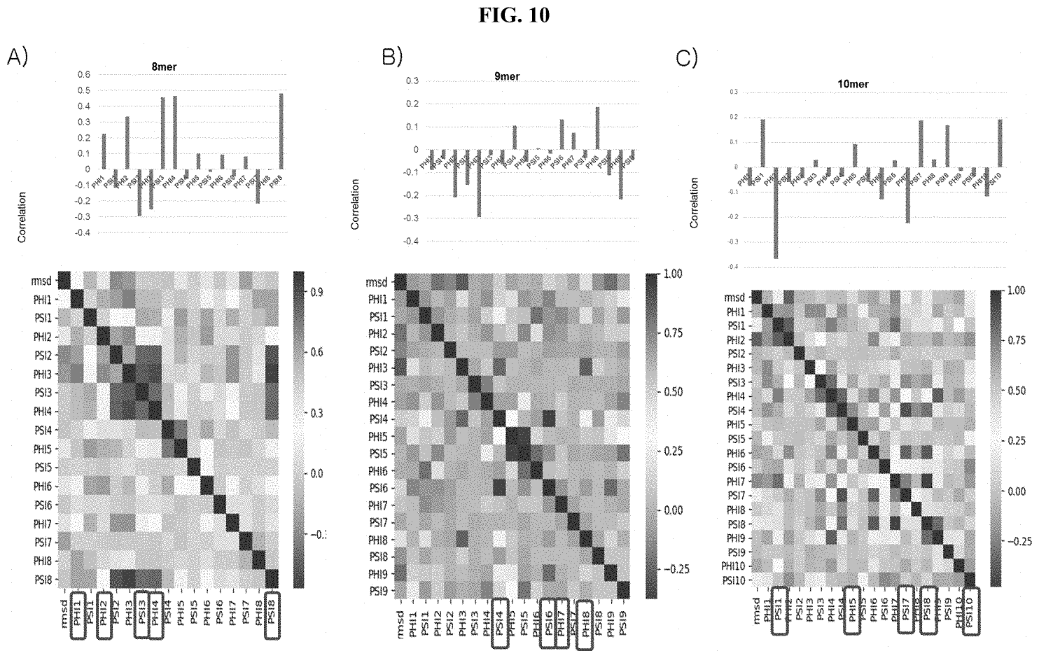

FIG. 10 shows the correlation between phi-psi angle and structure rmsds. FIG. 10 relates to step (53) which is the third step of IBA calculation, and shows the difference (rmsd: root mean square deviation) between all amino acid positions of peptides 8-mer, 9-mer and 10-mer and the docking structures.

As shown in FIG. 10, in the case of 8-mer, phi1, phi2, psi3, phi4 and psi8 showed high correlation, and in the case of 9-mer, psi4, psi6, phi7 and phi8 showed high correlation, and in the case of 10-mer, psi1, phi5, psi7, psi8 and psi10 showed high correlation.

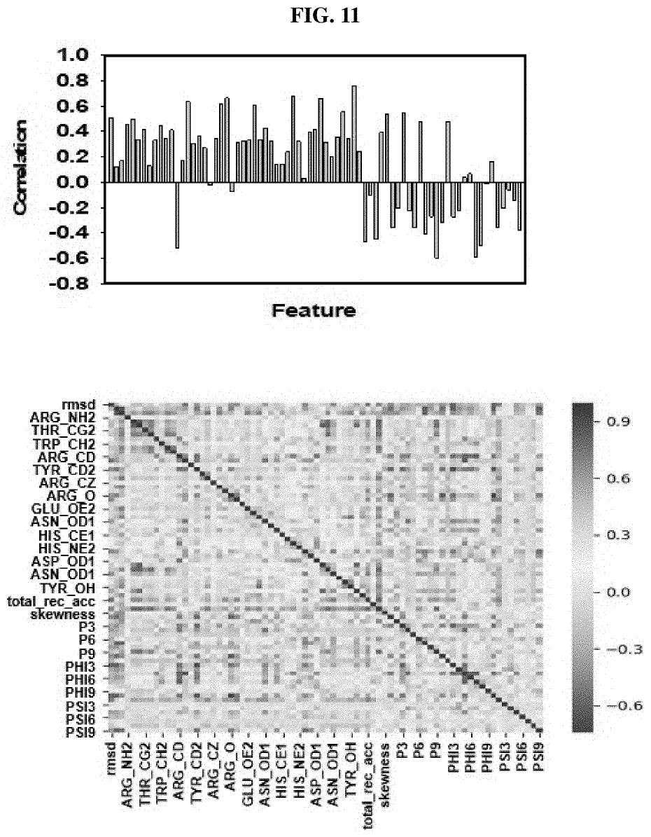

Meanwhile, FIG. 11 shows the correlation between the selected features and structure rmsds.

That is, FIG. 11 relates to step (54) which is the fourth step of IBA calculation, and shows the difference (rmsd: root mean square deviation) between the binding features of atoms based on moving snapshots of the selected amino acid positions and atoms of peptides and the docking structures. Here, high correlation between many features (correlation=0.8 to 1.0) is found.

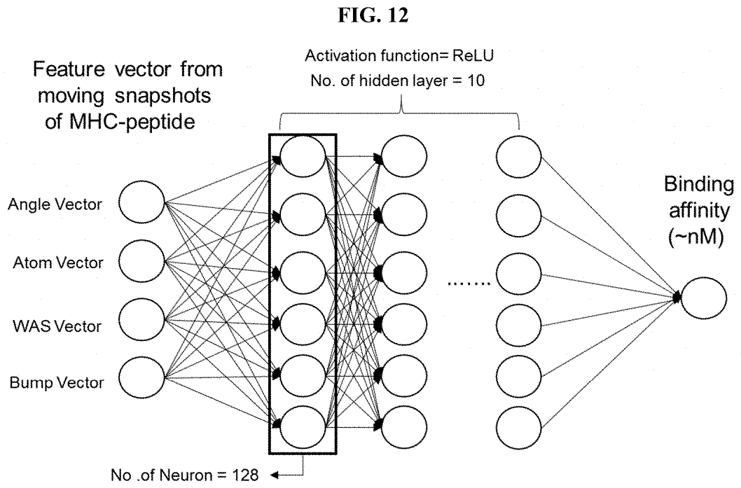

FIG. 12 shows a feature-based AI model structure generated from MHC-peptide complexes.

That is, FIG. 12 relates to step (55) which is the final step of IBA calculation, and shows the process of deep learning using features based on moving snapshots of the selected amino acid positions and atoms of peptides, atomic water accessible surface (WAS), and binding atom number.

Here, 10 hidden layers were used, and 128 neurons were used.

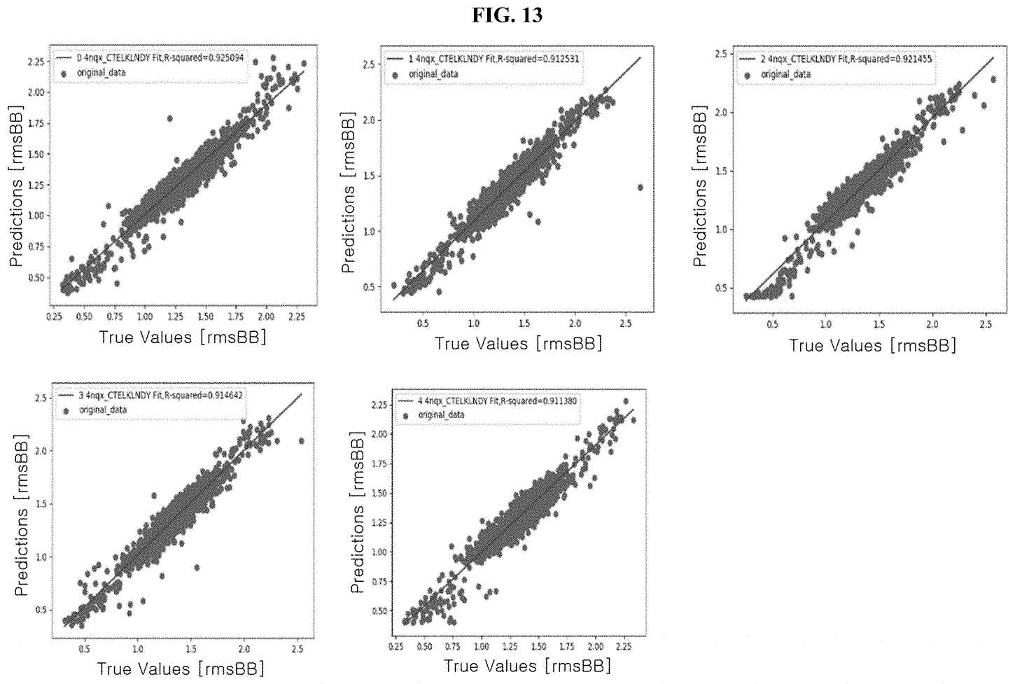

FIG. 13 shows the results of AI deep learning between the selected features and structure rmsds according to the present disclosure.

That is, the five figures of FIG. 13 show the R\hat{ }2 results of 5-fold cross-validation. In each of the five figures, rmsd<1 represents a region in which binding between the peptide and the MHC protein occurred well. Here, the x-axis indicates the rmsd values of the predicted structures, and the y-axis indicates the rmsd values of the known structures.

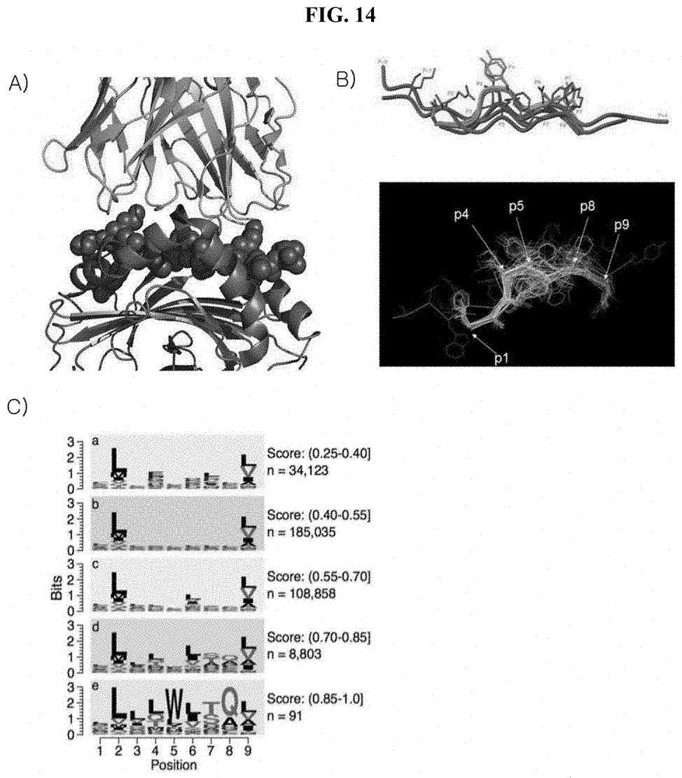

Meanwhile, FIG. 14 shows an example in which TCR activity is ranked.

That is, FIG. 14 shows the specific process of the sixth step which is the final step of the neoantigen-based personalized treatment method.

FIG. 14(A) shows an example in which TCR binds to the MHC-peptide.

FIG. 14(B) shows that the positions of about 100 different peptides that bind to the same HLA type overlap. In particular, p4, p5, p8 and p9 have patterns. In particular, p4, p5 and p8 are prominent, whereas p9 is buried inward.

FIG. 14(C) shows the pattern of position-specific TCR activity (Armen et. al, Frontiers in immunology, 2019). Thus, according to the method suggested by Armen, TCR is activated depending on the specific amino acids at specific positions according to HLA type.

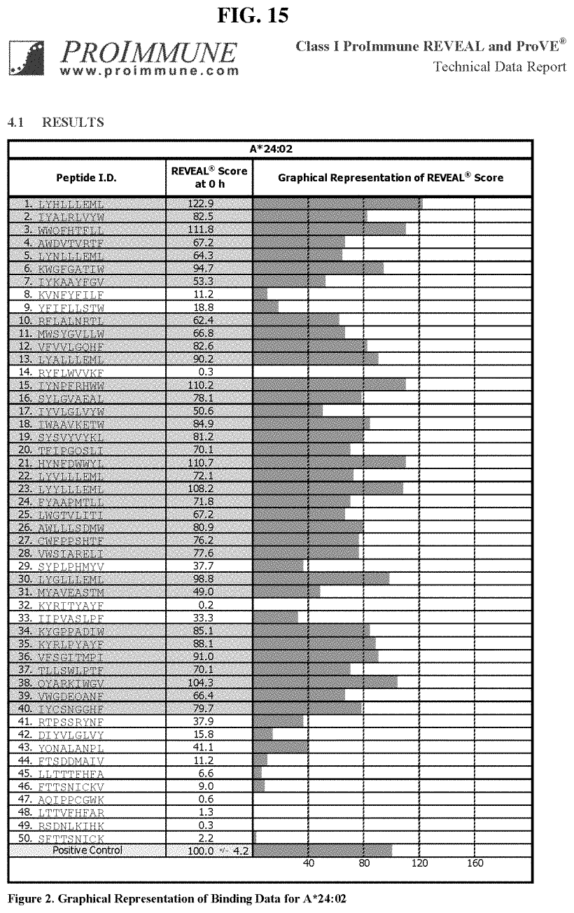

Meanwhile, FIG. 15 shows the results of verifying the in silico binding affinities (IBA) of predicted neoantigens for HLA-A*2402 by PROIMMUNE (testing institution).

As shown in FIG. 15, as peptides 1 to 40, those predicted as positive controls having in silico binding affinity (IBA) were used and evaluated, and as peptides 41 to 50, those predicted as negative controls having no binding affinity were used.

In addition, as activity >40 is evaluated as good binding, FIG. 15 shows that in silico binding affinity (IBA) of about 80% or more was successfully predicted.

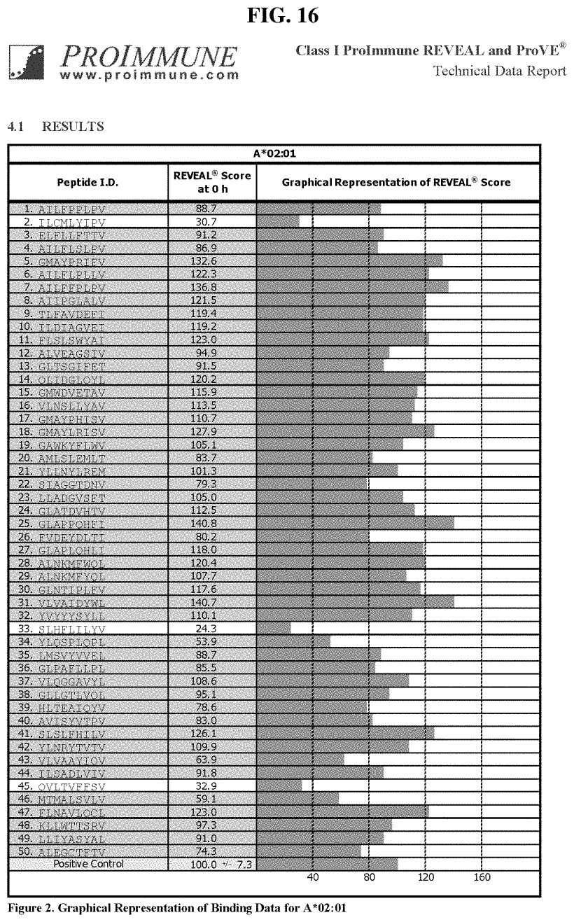

FIG. 16 shows the results of verifying the in silico binding affinities (IBA) of predicted neoantigens for HLA-A*0201 by PROIMMUNE (testing institution).

As shown in FIG. 16, as peptides 1 to 50, those predicted as positive controls having in silico binding affinity (IBA) were used and evaluated. As activity >40 is evaluated as good binding, FIG. 16 shows that in silico binding affinity (IBA) of about 90% or more was successfully predicted.

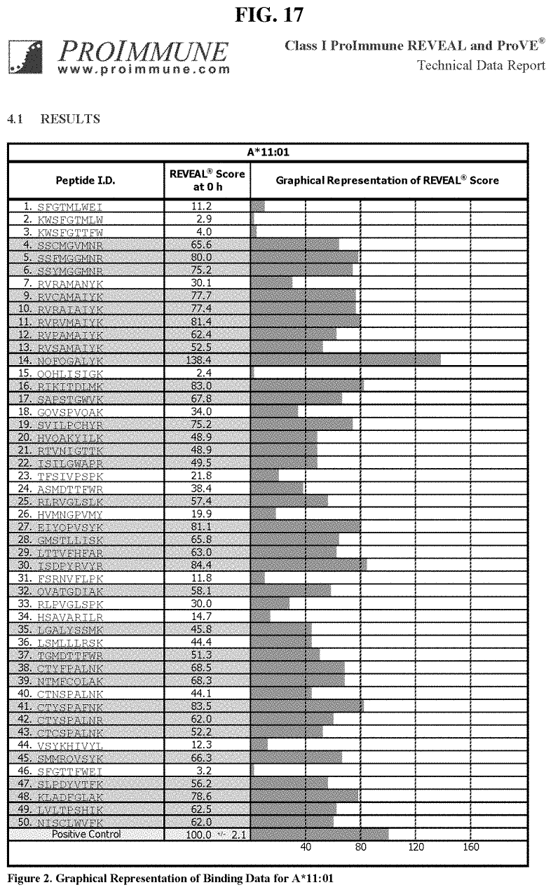

FIG. 17 shows the results of verifying the in silico binding affinities (IBA) of predicted neoantigens for HLA-A*11:01 by PROIMMUNE (testing institution).

As shown in FIG. 17, as peptides 1 to 50, those predicted as positive controls having in silico binding affinity (IBA) were used and evaluated. As activity >40 is evaluated as good binding, FIG. 17 shows that in silico binding affinity (IBA) of about 90% or more was successfully predicted.

As described above, the present disclosure relates to a system and a method of predicting neoantigens and induction of immune response based on molecular dynamics, the system and the method making it possible to verify induction of immunity against neoantigens having high binding affinity by identifying neoantigen candidates through genomic mutations and then predicting the binding affinities of the neoantigen candidates for MHC through the molecular dynamics. The present disclosure may contribute to the medical industrialization of patient-specific neoantigen prediction technology as AI deep learning-combined precision medical technology using big data.

According to the present disclosure as described above, the following effects may be expected.

That is, the present disclosure may contribute to the medical industrialization of patient-specific neoantigen prediction technology as AI deep learning-combined precision medical technology based on big data.

In addition, according to the present disclosure, neoantigens predicted based on NEOscan may be used for immunity induction verification tests, immunotherapy products, and cell therapy products based on T cell receptor-modified T (TCR-T) cells, chimeric antigen receptor-modified T (CAR-T) cells and tumor-infiltrating T lymphocytes (TIL). Thus, the present disclosure may also contribute to the development of therapeutic agents against diseases (including cancer) or phenotypes caused by inactivation or abnormalities of autoimmune systems.

The scope of the present disclosure is not limited to the embodiments described above, but is defined by the appended claims. It is obvious that those skilled in the art can make various modifications and alterations within the scope of the claims.

1. A method of providing neoantigen immunotherapy information for identifying a neoantigen using artificial intelligence (AI)-based molecular dynamics big data, the method comprising steps of:
(A) identifying neoantigen candidates through a genomic mutation;
(B) filtering the specificities of the neoantigen candidates for tissue and disease;
(C) predicting the in silico binding of the neoantigens to MHC; and
(D) calculating and ranking TCR activity.

2. The method of claim 1, wherein the genomic mutation is a mutation present in tumor exomes or tumor transcriptomes.

3. The method of claim 2, wherein the genomic mutation is any one of neo-mutations, exposed features or mal-functions, and verification of exome and transcriptome expression is performed by determining over-expression or differential expression in the transcriptome.

4. The method of claim 1, wherein the neoantigen candidates in step (A) comprise any one or more of major clone genes selected from cancer cells, mesenchymal stroma cell (MSC) genes selected from cancer cells, or six HLA types of cancer cells.

5. The method of claim 4, wherein the six HLA types are HLA-A, HLA-B, HLA-C, HLA-DR, HLA-DP and HLA-DQ.

6. The method of claim 4, wherein, for selecting the major clone genes from cancer cells, a clone having the largest number of cancer cells is selected as a major clone from cancer cells composed of the major clone and subclones.

7. The method of claim 4, wherein the mesenchymal stroma cell (MSC) genes selected from cancer cells are collected based on somatic mutation of genes expressed in the stroma cells.

8. The method of claim 4, wherein the HLA types of the cancer cells are selected through genomic HLA typing.

9. The method of claim 4, wherein determination of the HLA types of the cancer cells is performed by a method comprising steps of:
(a1) collecting read sequences of HLA genes;
(a2) aligning the HLA gene read sequences to a human reference genome sequence according to allele types; and
(a3) determining the types of the HLA genes according to the aligned ranks of the HLA genes.

10. The method of claim 1, wherein step (B) is performed by determining a tissue in which the neoantigen candidates are expressed.

11. The method of claim 1, wherein the predicting of the in silico binding in step (C) is performed by calculating in silico binding affinities (IBA) based on the three-dimensional structures of peptides based on somatic mutation of tissue-specific genes, produced through steps (A) and (B), and a selected MHC protein.

12. The method of claim 11, wherein the predicting of the in silico binding in step (C) is performed by producing peptides based on somatic mutation of the specific genes, and calculating the in silico binding affinities (IBA) through docking based on the three-dimensional structures of the produced peptides and the MHC protein.

13. The method of claim 11, wherein the predicting of the in silico binding in step (C) is performed by generating binding models for a number of types of antigens and calculating the energy difference and RMSD difference therebetween.

14. The method of claim 11, wherein the predicting of the in silico binding in step (C) is performed by a method comprising steps of:
(C1) performing dynamics simulation for MHC-peptide docking complexes;
(C2) generating a phi-psi angle Ramachandran plot based on MHC-peptide docking data;
(C3) calculating the correlation between rmsds through the phi-psi angles and structures;
(C4) calculating the correlation between selected features and each structure rmsd; and
(C5) determining the in silico binding affinities through an AI model based on features generated from MHC-peptide complexes.

15. The method of claim 11, wherein the in silico binding affinity (IBA) is calculated by the ratio of a predicted drug response (ic50) of a mutant gene to a predicted drug response (ic50) of a wildtype gene.

16. A system of providing neoantigen immunotherapy information for identifying a neoantigen using AI-based molecular dynamics big data, the system being configured to provide the neoantigen immunotherapy information by identifying neoantigen candidates through a genomic mutation, filtering the specificities of the neoantigens for tissue and disease, predicting the in silico binding of the neoantigens to MHC, and then calculating TCR activity.

17. The system of claim 16, wherein the neoantigen candidates comprise any one or more of major clone genes selected from cancer cells, mesenchymal stroma cell (MSC) genes selected from cancer cells, or six HLA types of cancer cells.

18. The system of claim 17, wherein, for selecting the major clone genes from cancer cells, a clone having the largest number of cancer cells is selected as a major clone from cancer cells composed of the major clone and subclones.

19. The system of claim 17, wherein the mesenchymal stroma cell (MSC) genes selected from cancer cells are collected based on somatic mutation of genes expressed in the stroma cells.

20. The system of claim 17, wherein the six HLA types of the cancer cells are selected through genomic HLA typing.

21. The system of claim 17, wherein determination of the HLA types of the cancer cells is performed by a method comprising steps of:
(a1) collecting read sequences of HLA genes;
(a2) aligning the HLA gene read sequences to a human reference genome sequence according to allele types; and
(a3) determining the types of the HLA genes according to the aligned ranks of the HLA genes.

22. The system of claim 16, wherein the filtering of the specificity of the neoantigen candidates is performed by determining a tissue in which the neoantigen candidates are expressed.

23. The system of claim 16, wherein the predicting of the binding is performed by calculating in silico binding affinities (IBA) based on the three-dimensional structures of produced peptides based on somatic mutation of tissue-specific genes and a selected MHC protein.

24. The system of claim 23, wherein the predicting of the binding is performed by producing peptides based on somatic mutation of the specific genes, and calculating the in silico binding affinities (IBA) through docking based on the three-dimensional structures of the produced peptides and the MHC protein.

25. The system of claim 24, wherein the predicting of the binding is performed by a method comprising steps of:
(C1) performing dynamics simulation for MHC-peptide docking complexes;
(C2) generating a phi-psi angle Ramachandran plot based on MHC-peptide docking data;
(C3) calculating the correlation between rmsds through the phi-psi angles and structures;
(C4) calculating the correlation between selected features and each structure rmsd; and
(C5) determining the in silico binding affinities through an AI model based on features generated from MHC-peptide complexes.

26. The system of claim 23, wherein the in silico binding affinity (IBA) is calculated by the ratio of a predicted drug response (ic50) of a mutant gene to a predicted drug response (ic50) of a wildtype gene.
